(19) United States
(12) Patent Application Publication
(10) Pub. No.: US 2026/0056151 A1
(43) Pub. Date: Feb. 26, 2026

(54) Determination of Material Composition Using Compton Scatter

(71) Applicant: Viken Detection Corporation, Burlington, MA (US)

(72) Inventor: Peter J. Rothschild, Newton, MA (US)

(21) Appl. No.: 19/259,729

(22) Filed: Jul. 3, 2025

Related U.S. Application Data
(60) Provisional application No. 63/667,436, filed on Jul. 3, 2024.

(51) Int. Cl. G01N 23/2206; G01N 23/20066; G01N 23/223; G01N 33/202

(52) U.S. Cl. CPC G01N 23/2206; G01N 23/20066; G01N 23/223; G01N 33/202; G01N 2223/205; G01N 2223/301; G01N 2223/628

(57) ABSTRACT

A method of determining material composition of a target includes identifying a component of the material composition of the target by matching a Compton scattering background, in a spectrum of detected X-rays acquired by irradiating the target with a source of X-rays during a nominal source exposure time period, to a Compton scattering spectrum for the component; and reporting the material composition of the target including the identified component of the material composition.

### RELATED APPLICATION

This application claims the benefit of U.S. Provisional Application No. 63/667,436, filed on Jul. 3, 2024. The entire teachings of the above application are incorporated herein by reference.

### BACKGROUND

X-ray fluorescence (XRF) is a well-known technique that can be used to identify the elemental composition of materials. An X-ray source, such as a radioactive isotope or an X-ray tube, can be used as a source of X-rays that irradiate the sample to be inspected, and an energy-resolving detector can be used to detect the characteristic fluorescence X-rays emitted by the material upon excitation. For example, lead will emit L-shell X-rays with energies of 10.5 keV and 12.6 keV, and K-shell X-rays at energies of 75 keV and 85 keV. By detecting these specific X-ray fluorescence lines, the presence of lead can be determined, and the higher the intensity of the X-ray lines, the larger the concentration of lead that is present.

### SUMMARY

Example embodiments include an apparatus, corresponding method, and non-transitory computer-readable medium for determining elemental composition of a target, such as a pipe. An outer housing may be configured to be positioned against, or close to, the pipe so that a measurement can be acquired. For buried pipes, an excavation hole can be created to allow proximal access to the pipe, and an outer housing may be configured to be positioned against, or close to, the pipe via a rigid insertion member, such as a rod or pole. A radioactive X-ray source may be encompassed by a shield within the housing. A shutter may be coupled to the shield and configured to open selectively to enable radiation from the X-ray source to exit the shield and irradiate an inner wall of the pipe. An X-ray detector within the housing may be configured to detect fluorescence X-rays emitted from the pipe upon irradiation by the X-ray source. A controller may be communicatively coupled to the X-ray detector and configured to determine the presence of one or more elemental materials contained within the pipe based on the fluorescence X-rays.

The elemental materials determined by the controller may include lead. The radioactive X-ray source may include Co-57 and/or Cd-109. Alternatively, an X-ray tube may be the source of irradiating radiation, with the end-point energy of the source being above approximately 25 keV to produce L-shell fluorescence from lead contained within the pipe, and preferably, above approximately 100 keV to produce more highly penetrating K-shell fluorescence from lead contained within the pipe. The X-ray detector may include one or more of a silicon drift detector, a silicon photodiode, cadmium telluride, and cadmium zinc telluride.

In addition to the detection of XRF produced from elements within the pipe, the irradiating radiation from the radioactive source or X-ray tube within the housing can undergo Compton scattering, and the Compton-scattered X-rays can be detected by the X-ray detector within the housing. These scattered X-rays form a background lying under any of the detected fluorescence X-ray peaks. A shape of the energy spectrum of the detected Compton scattered X-rays can then be analyzed to provide additional information regarding a material composition of the pipe, and optionally, regarding a diameter of the pipe. This additional information is helpful for pipes composed of lower atomic number elements, such as plastic, copper, iron, galvanized steel, brass, or a combination. The elements present in these pipes produce fluorescence X-rays with such low energy that they are more difficult to detect reliably. For example, the highest-energy fluorescence X-rays from iron and copper are 7.1 keV and 8.9 keV, respectively. These very low-energy X-rays can be easily absorbed by corrosion deposits or soil that remains on the outside of the pipe, meaning that cleaning of the pipe becomes critical to correct identification of the pipe composition. The Compton-scattered X-rays, on the other hand, can have a much higher energy. For example, for a Co-57 radioactive isotope source, the average energy of the scattered X-rays from steel is 95 keV, and therefore the scattered X-rays are largely unaffected by the condition and cleanliness of the pipe surface.

In one preferred embodiment, the shape of the energy spectrum of the detected Compton-scattered X-ray background of the energy spectrum can be used to determine the base composition of the pipe. The Compton scatter background typically includes a large peak, called the “Compton peak” at the higher-energy end of the spectrum, with a lower-energy tail. Position, shape, and width of the Compton peak are all unique to the material composition of the object from which the X-rays are Compton scattered. For example, the Compton peaks for scatter from PVC plastic, aluminum, and steel are shown in FIG. 1. The Compton peak on the high-energy side (right side, in FIG. 1) of the spectrum has a different shape and height for each of the materials. By finding a best fit of the measured Compton scatter background to a library of background spectra (a set of reference Compton scatter spectra for a set of respective materials), the composition of the target material can be determined with a high degree of confidence.

In a first particular embodiment, a method of determining material composition of a target includes:
a) identifying a component of the material composition of the target by matching a Compton scattering background, in a spectrum of detected X-rays acquired by irradiating the target with a source of X-rays during a nominal source exposure time period, to a template Compton scattering spectrum for the component;
b) producing a lead fluorescence spectrum for the target by using the template Compton scattering spectrum to remove the Compton scattering background from the spectrum of detected X-rays;
c) calculating a proportion of lead in the target by using the Compton scattering background, the lead fluorescence spectrum, and the nominal source exposure time period; and
d) reporting the material composition of the target including the identified component of the material composition and the proportion of lead in the target.

In a second particular embodiment, a method of determining material composition of a target includes:
a) identifying a component of the material composition of the target by matching a Compton scattering background, in a spectrum of detected X-rays acquired by irradiating the target with a source of X-rays during a nominal source exposure time period, to a Compton scattering spectrum for the component; and
b) reporting the material composition of the target including the identified component of the material composition.

In a third particular embodiment, a non-transitory computer-readable storage medium stores instructions that, when executed by a computer, cause the computer to perform a method for colorizing an output X-ray image of a target, the method comprising the elements of the first particular embodiment.

In a fourth particular embodiment, a non-transitory computer-readable storage medium stores instructions that, when executed by a computer, cause the computer to perform a method for colorizing an output X-ray image of a target, the method comprising the elements of the second particular embodiment.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a graph showing example energy spectra of Compton scatter from some common materials.

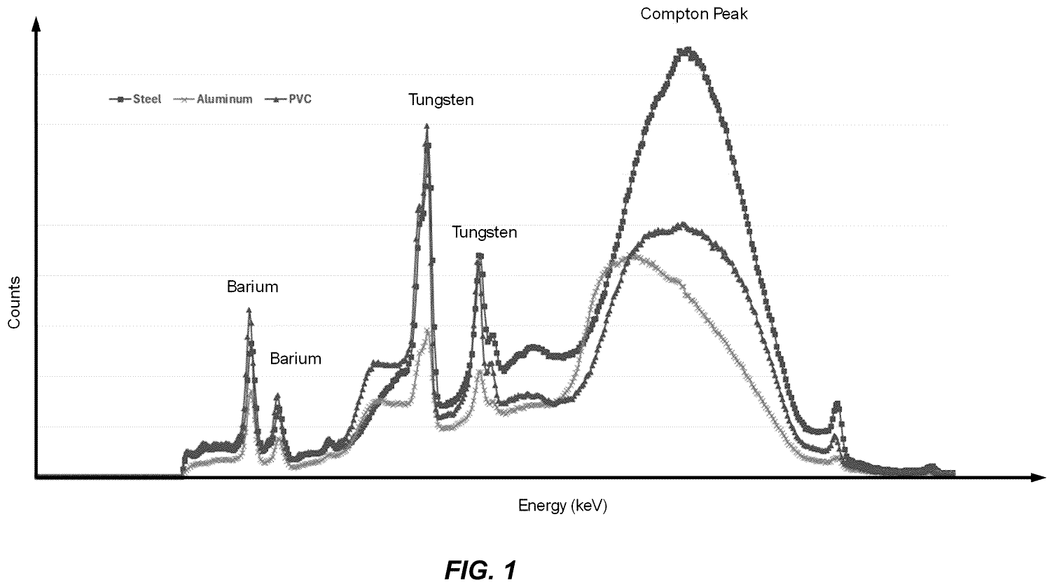

FIG. 2 illustrates components of a preferred embodiment of a device for determining material composition of pipes.

FIG. 3 is a flow diagram illustrating a process of identifying a material and elemental composition of a target object according to an embodiment.

FIG. 4. Use of an extender pole with an embodiment of the device to access a buried pipe.

FIG. 5. Example energy spectra showing the Compton scatter background and fluorescence peaks from some common pipe materials.

FIG. 6. Example energy spectra showing the Compton scatter background and lead and bismuth fluorescence peaks from brass and non-lead brass pipe material.

FIG. 7. The output of a preferred embodiment of an algorithm that can simultaneously fit lead and bismuth fluorescence peaks to an energy spectrum.

FIG. 8 is a block diagram illustrating an example computer or digital processing environment in which methods of determining material composition according to various embodiments may operate.

FIG. 9 is a flow diagram illustrating a method of determining material composition according to an embodiment.

FIG. 10 is a flow diagram illustrating a method of determining material composition according to an alternative embodiment.

### DETAILED DESCRIPTION

### General Considerations

The figures and descriptions provided herein may have been simplified to illustrate aspects that are relevant for a clear understanding of the herein described devices, systems, and methods, while eliminating, for the purpose of clarity, other aspects that may be found in typical similar devices, systems, and methods. Those of ordinary skill may recognize that other elements and/or operations may be desirable and/or necessary to implement the devices, systems, and methods described herein. But because such elements and operations are well known in the art, and because they do not facilitate a better understanding of the present disclosure, a discussion of such elements and operations may not be provided herein. However, the present disclosure is deemed to inherently include all such elements, variations, and modifications to the described aspects that would be known to those of ordinary skill in the art.

The terminology used herein is for the purpose of describing particular example embodiments only and is not intended to be limiting. For example, as used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

Although the terms first, second, third, etc., may be used herein to describe various elements, components, regions, layers and/or sections, these elements, components, regions, layers and/or sections should not be limited by these terms. These terms may be only used to distinguish one element, component, region, layer or section from another element, component, region, layer or section. That is, terms such as “first,” “second,” and other numerical terms, when used herein, do not imply a sequence or order unless clearly indicated by the context.

### Definitions

As used in this description and the accompanying claims, the following terms shall have the meanings indicated, unless the context otherwise requires:

“Set,”as used herein, includes at least one member.

“Illuminate” and “irradiate” and related words, when used herein in relation to source X-rays to impinging on a target or other surface, mean the same thing.

A description of example embodiments follows.

Recently, the issue of lead contamination of water supplies in the United Stated has become even more important, especially following the widespread contamination of the water supply in Flint, Michigan. In this instance, the water delivery infrastructure contained many sections of old lead service lines (LSLs), and changes in the chemicals being used to treat the water caused leaching of significant quantities of lead into the water supply, causing lead poisoning in many young and vulnerable children.

Recently, the United States Federal Government has approved many billions of dollars to start removing LSLs from the water supply. This process involves locating the presence of LSLs, many of which are buried at depths of 3-6 feet underground, and replacing them with non-toxic pipes such as plastic, copper, or galvanized steel.

Due to the depth at which they are buried, identifying and locating buried lead pipes presents a substantial challenge. One existing approach is to use vacuum excavation to gain access to the underground pipe for testing. Once access is obtained, it is possible to determine by visual inspection whether the pipe is a lead pipe or of some other material. However, visual inspection is often difficult due to corrosion of the outside of the pipe, and it will not reveal hidden lead, such as a lead content of brass fittings, or a use of a lead liner in a galvanized steel pipe. Another method, which is often not practical due to the location of the water meter at a residence, is an inspection of pipes that are directly connected to a meter, such as a water meter. However, while such connecting pipes are often made of a non-lead material, buried or concealed service lines connected thereto may still contain lead.

X-ray fluorescence (XRF) is a well-known technique that can be used to identify the elemental composition of materials. An X-ray source such as a radioactive isotope or an X-ray tube can be used to create X-rays that irradiate the sample to be inspected, and an energy-resolving detector can be used to detect the characteristic fluorescence X-rays emitted by the material upon excitation. For example, lead will emit L-shell X-rays with energies of 10.5 keV and 12.6 keV, and K-shell X-rays at energies of 75 keV and 85 keV. By detecting these specific X-ray fluorescence lines, the presence of lead can be determined, and the higher the intensity of the X-ray lines, the larger the concentration of lead that is present.

As an example, there are existing lead analyzers that are designed specifically for lead paint inspection. One such device, the Viken Detection® Pb200i, uses a Co-57 radioactive isotope to excite the lead atoms in the paint, and the device detects the K-Alpha X-rays at 75 keV. These X-rays have sufficient energy to penetrate any overlying paint that may not contain lead, thereby allowing the levels of even deeply buried lead to be accurately measured. In contrast, the L-shell X-rays with their much lower energies may not be able to penetrate the overlying paint, giving erroneous results.

Another example specifically designed for inspecting the material composition of buried pipes using an X-ray fluorescence device is described in issued patent U.S. Pat. No. 11,796,494 titled “Compact Insertable X-Ray Fluorescence Device for Pipe Inspection,” which is hereby incorporated herein by reference in its entirety.

Example embodiments described herein include a compact XRF analyzer device that can positioned onto, or next to, a pipe, allowing the materials making up the pipe walls to be identified. The device can be held against the pipe by hand during a measurement, if access to the pipe permits such a measurement. If the pipe is deeply buried as is often the case, and the access hole to the pipe has a small diameter, then the device can be mounted on an extender pole that allows the device to be inserted down the hole and positioned onto the exposed pipe wall.

Such an extender pole typically has a trigger mechanism that allows the operator to activate the device remotely, from the distal (user) end of the pole. Such embodiments enable the presence of lead pipes, or pipes containing lead, to be detected, provided that the device can be positioned onto, or close to, an exposed section of the pipe. Devices in example embodiments may contain a radioactive source due to its small size and extremely rugged structure. This configuration contrasts with X-ray sources that require a high-voltage generator in the instrument close to the X-ray source, and which is vulnerable to shock, adverse environmental conditions, and prone to failure. Alternative embodiments can contain an X-ray source such as a miniature X-ray tube in place of a radioactive source.

A particular embodiment device, which is also referred to herein as an apparatus for determining the elemental composition of a pipe, includes: an outer housing that can be positioned onto, or next to, a pipe under inspection; a radioactive source of X-rays contained within shielding material within the housing; a shutter that can be remotely opened and closed such that unobstructed radiation can irradiate the outer wall of the pipe; an X-ray detector located within the housing that detects scattered X-rays and fluorescence X-rays emitted from the pipe wall upon irradiation with the source; a controller that analyzes the Compton scatter background and indicates the presence of one or more composition materials contained within the pipe wall; and a controller that analyzes the fluorescence peaks and indicates the presence of one or more elemental materials contained within the pipe wall.

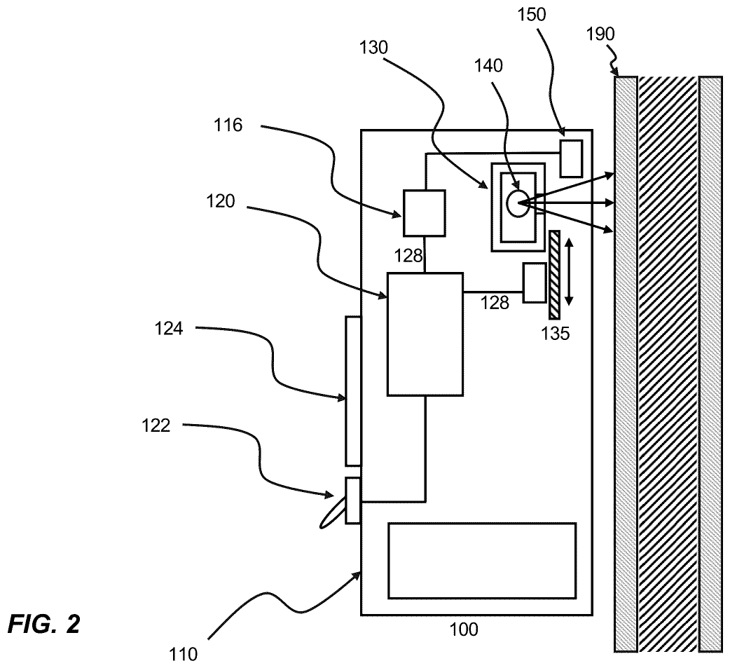

FIG. 2 is a schematic illustration of a device 100 for determining elemental composition of a pipe 190 in one embodiment. The outer housing 110 may be composed entirely of a material such as PVC plastic, vinyl, rubber, polyurethane or aluminum.

The outer housing 110 may house a radioactive X-ray source 140 that is encompassed by a shield 130. For example, X-ray source 140 may include Co-57 and/or Cd-109, which emit radiation that is blocked from entering the surrounding environment by radiation shield 130. However, when the device 100 is inserted next to a pipe to be inspected, a shutter 135 coupled to the shield 130 may selectively open to enable radiation from the X-ray source 140 to exit the shield 130 and irradiate an outer wall of the pipe 190. For example, shutter 135 may be positioned within or adjacent to an opening in a wall of the shield 130. An X-ray detector 150 within housing 110 may then detect scattered X-rays and fluorescence X-rays emitted from the pipe upon irradiation by the X-ray source 140. The X-ray detector 150 may include, for example, a silicon drift detector, a silicon photodiode, a cadmium-telluride (CdTe) detector and/or a cadmium-zinc-telluride (CdZnTe) detector. The X-ray detector 150 may be cooled via a cooling mechanism (not shown) to provide higher energy resolution, allowing the detection limits of the lead or other materials to be lowered. An example X-ray detector 150 may be a small CdTe detector in a TO-8 can, which may have an active diameter of about 5 mm and contain a piezo-electric cooler contained within the can. Such a detector can be very compact, with an exterior diameter of about 15 mm, making it suitable for installation within a small compact outer housing.

A controller 120 may be communicatively coupled to the X-ray detector 150 and the shutter 135 via power and I/O cables 128. Controller 120 may operate to control the opening and closing of the shutter 135 and process data from the X-ray detector 150 to determine the material composition of the pipe from the shape of the Compton scatter background. Additionally, the controller may process data from the X-ray detector to determine the presence of one or more elemental materials contained within the pipe based on the fluorescence X-rays detected by the X-ray detector 150. A preamplifier and the high voltage power supply (HVPS) 116 may be connected between the controller 120 and the X-ray detector 150. The preamplifier and HVPS 116 may provide, for example, a ˜600V biasing voltage for the detector. The controller 120 may have a trigger or activation (“acquire”) switch 122 to actuate the opening of the shutter 135 and activate the X-ray detector 150 to initiate the collection of data, thereby causing the X-ray source 140 to irradiate the exterior wall of the pipe and cause the X-ray detector 150 to detect X-rays Compton-scattered from the pipe under inspection, and to detect corresponding fluorescence X-rays emitted from the pipe.

A display 124 can indicate the material composition of the pipe, and optionally, a level of elemental material(s) (e.g., lead) detected by the X-ray detector 150. The controller 120 may include a wireless interface (not shown) to communicate wirelessly with another computing device (e.g., laptop, smartphone, workstation) to transmit data indicating the material composition of the pipe, and optionally, the level of elemental material(s) detected, and/or may receive and act upon commands from the computing device to operate the device 100 and acquire readings of the pipe.

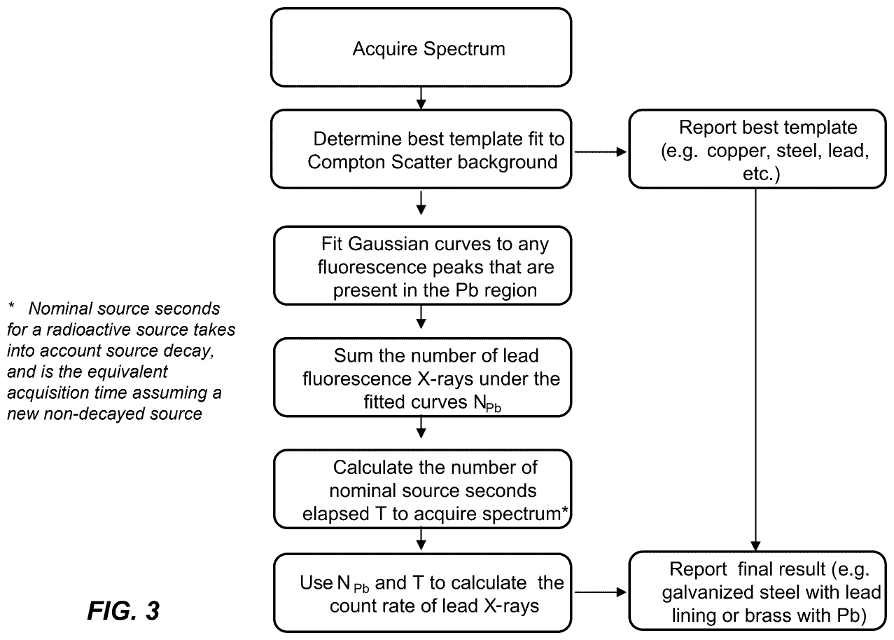

FIG. 3 is a flow diagram of a process 300 of determining material composition and optionally, the elemental composition, of a pipe in one embodiment. Remediation of an LSL to a dwelling may involve determining if any lead pipe is present in the pipe between the water shutoff at the curb-stop near the water main and the water meter located on an exterior wall of the dwelling, or within the basement of the dwelling. Currently, the presence of the lead pipe must be inferred from 1) records, which typically are not available or reliable due to the age of the LSL, 2) gaining access to the LSL by excavating around the LSL, and performing a visual inspection, or 3) by noticing the presence of exposed lead pipes near the water meter. However, often the lead will not be visible even when present.

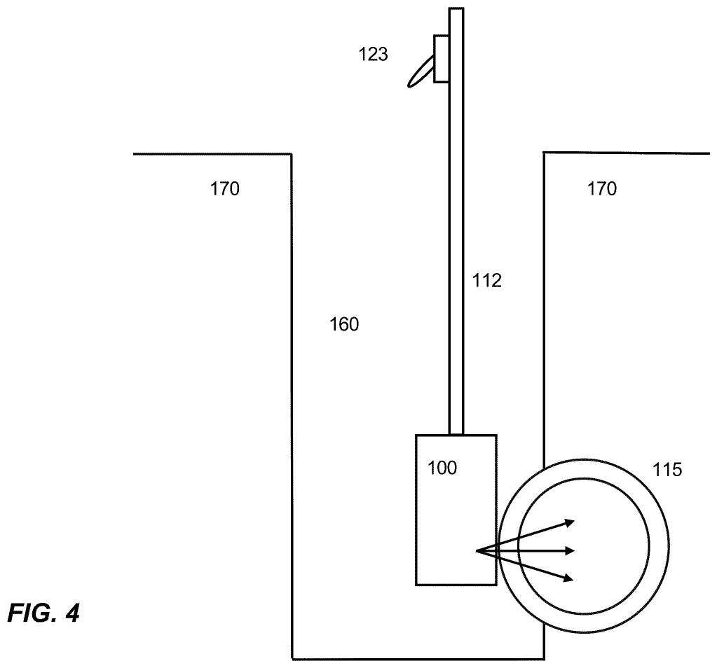

In contrast, example embodiments such as the device 100 may be employed in the process 300 described below to detect the presence of LSLs to a dwelling. With reference to FIG. 4, a small diameter excavation hole 160 in the ground 170 can be used to gain access to the buried WSL. Device 100 may then be inserted, using an extender pole 112, to position the device next to the wall of pipe 115. Once positioned against the pipe, device 100 may then be activated via the controller 120 to record pipe material composition, and optionally, lead readings, as described above. Based on the readings captured by the controller 120, the data can then be used to assess the lead content of the pipe and the best remediation method (325).

Controller 120 can be activated by the operator via a remote trigger switch 123 mounted on the extender pole. Remote trigger switch can be mechanically coupled to the trigger switch 122 mounted on device 100, or it can be coupled to controller 120 via electrical signals.

Alternatively, controller 120 can be activated to acquire a measurement via a remote controller, using Wi-Fi or Bluetooth radio signals.

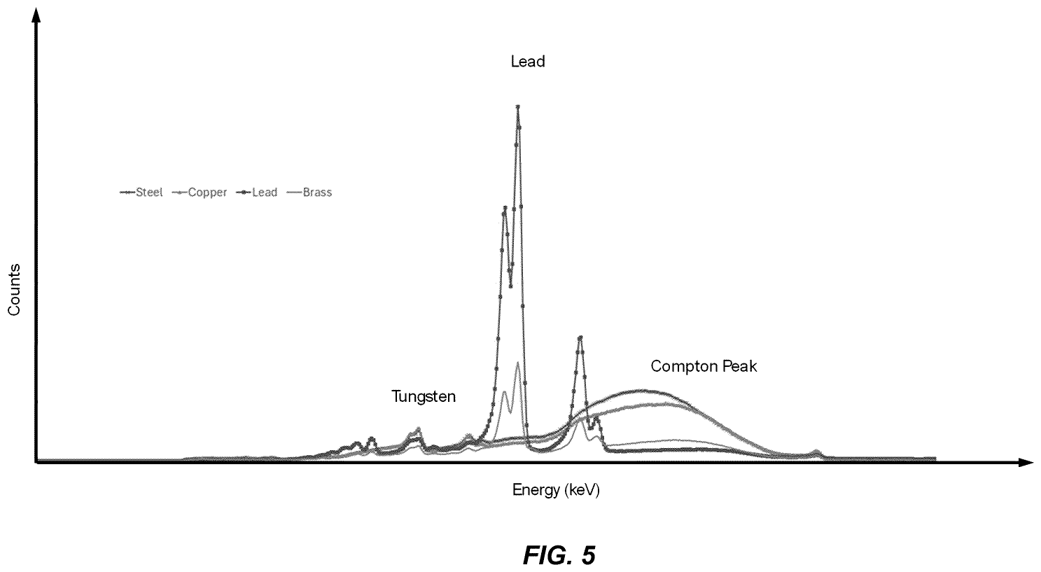

As previously described, the pipe material composition is best determined from the shape of the Compton scatter background in the energy spectrum of X-rays detected by detector 150 that lies beneath any fluorescence peaks emitted from the pipe that may be present. Example energy spectra containing Compton scatter backgrounds from common pipe materials are shown in FIG. 5. These spectra were created from a device containing a Co-57 radioactive isotope source. The spectrum containing the tallest lead fluorescence peaks corresponds to a pure lead pipe, while the spectrum containing the smaller lead fluorescence peaks corresponds to leaded brass. Also shown are the energy spectra for copper and steel pipes that contain no lead, and therefore have no measurable lead fluorescence peaks. The tungsten fluorescence peaks are emitted from the tungsten shielding surrounding the radioactive isotope source in the device.

In preferred embodiments of the device, the material composition of the pipe being inspected is determined by fitting a library or pre-stored templates corresponding to energy spectra of common pipe materials, such as copper, steel, brass, non-lead and lead-free brass, copper-zinc alloys, and lead. The templates can be sequentially fitted by scaling the amplitudes of the templates by the length of the acquisition time of the measurement. For example, if the pre-stored templates were acquired with a 100 second acquisition time, then for a 10 second measurement, the templates would first be scaled by a factor of 0.1, and the templates would then be further scaled by the fitting algorithm to most accurately fit the measured energy spectrum. Preferred embodiments of the fitting algorithm also allow the spectra to be scaled along the energy axis, to account for variations in the energy gain of detector 150 in device 100. The template that minimizes the chi-square value of the fit is selected to most accurately represent the material composition of the pipe under measurement. The chi-square value is representative of the quality of the match between the scaled template and the measure energy spectrum, and the smaller the chi-square value, the more accurate the fit.

In other embodiments, a library of pre-stored templates is not used. Instead, the shape and location of the Compton peak in the energy spectrum can be analyzed for characteristics such as centroid location, Full-Width Half Maximum (FWHM) of the Compton peak, and skewness of the Compton Peak (i.e. asymmetry of the Compton peak about its centroid). This is typically a more difficult approach to implement and has been found to be less effective.

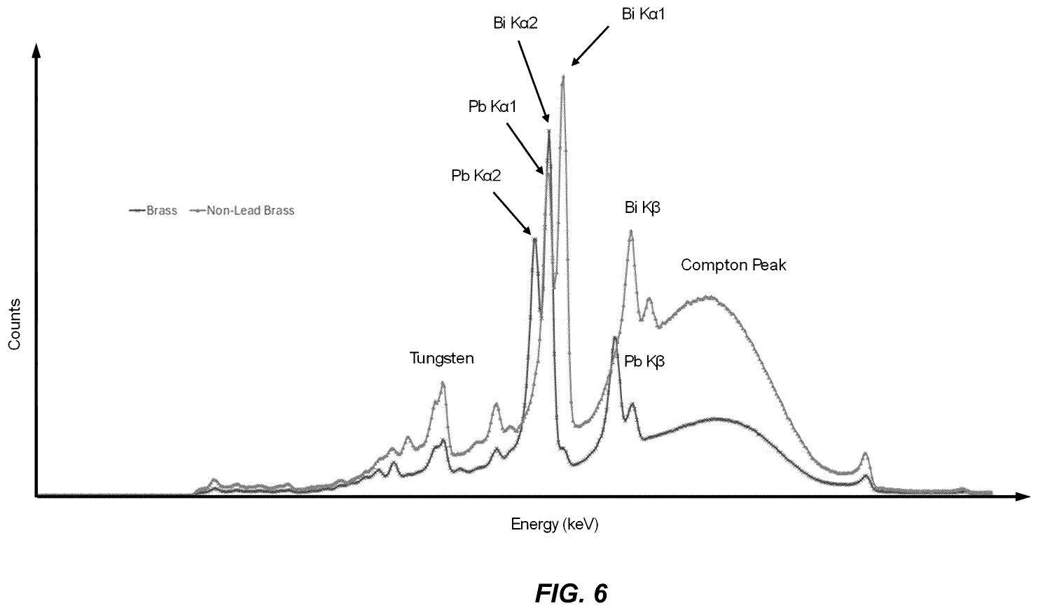

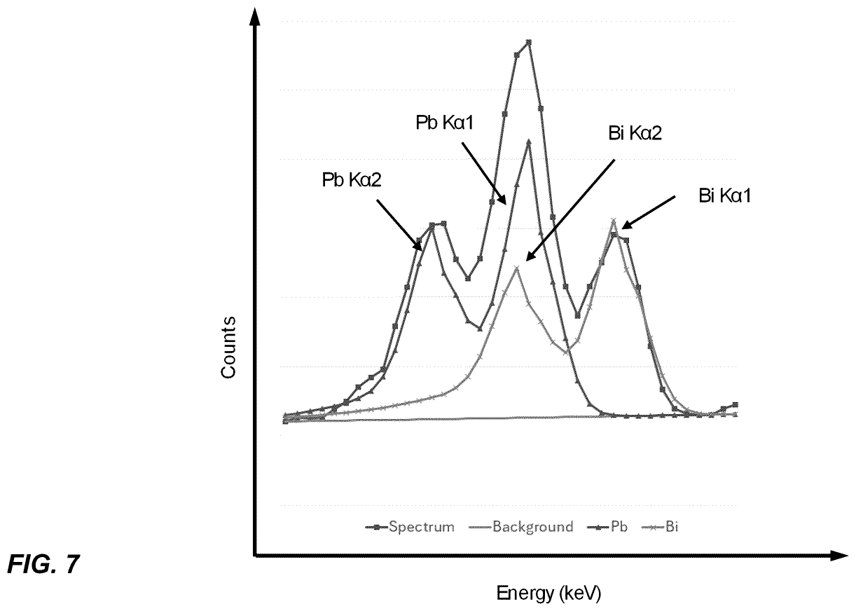

One of the complications of performing pipe analysis to determine lead content using X-ray fluorescence is that some type of low-lead or zero-lead brass contains bismuth as an alternative to lead. Because the atomic number of bismuth (Z=83) is only one greater than lead (Z=82), the fluorescence peaks lie very close to each other in the energy spectrum, and some of the peaks overlap with each other. Referring to FIG. 6, energy spectra from a brass pipe containing large levels of lead (Pb), and a non-lead brass pipe containing bismuth (Bi) instead of lead are shown. It can be seen that the Pb Kα1 peak and the Bi Kα2 peak overlap within the energy resolution of the detector. To accurately determine the lead content of the pipe, preferred embodiments of the analysis algorithm simultaneously fit lead and bismuth peaks to any excess fluorescence X-ray counts above the background in the energy region of the fluorescence peaks. This is shown schematically in FIG. 7, where a preferred algorithm has simultaneously fit Kα1 and Kα2 peaks for both lead and bismuth, so that the combined peaks summed with the fitted Compton scatter background best match the measured energy spectrum. The integrated area under the twin lead peaks and the twin bismuth peaks then yields the counts for lead and bismuth, separately. By dividing the counts by the time duration of the measurement, the count rate for lead can be accurately displayed, regardless of the bismuth content of the pipe.

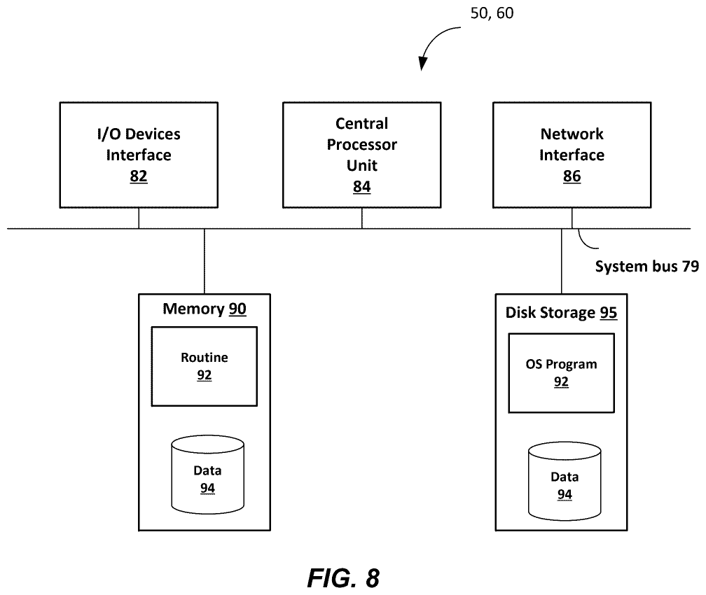

FIG. 8 is a block diagram illustrating an example computer or digital processing environment in which methods of determining material composition according to various embodiments may operate. Further, non-transitory computer-readable storage media according to various embodiments may be implemented in such an environment. FIG. 8 shows internal structure of a computer (e.g., client processor/device 50, server computer 60, or a computer in an X-ray scanning device such as the device illustrated in FIG. 2). Each computer 50, 60 contains system bus 79, where a bus is a set of hardware lines used for data transfer among the components of a computer or processing system. Bus 79 is essentially a shared conduit that connects different elements of a computer system (e.g., processor, disk storage, memory, input/output ports, network ports, etc.) that enables the transfer of information between the elements. Attached to system bus 79 is I/O device interface 82 for connecting various input and output devices (e.g., keyboard, mouse, displays, printers, speakers, buttons or other controls of an X-ray scanning device, etc.) to the computer 50, 60. Network interface 86 allows the computer to connect to various other devices attached to a network, which may be external to the components illustrated in FIG. 8. Memory 90 provides volatile storage for computer software instructions 92 and data 94 used to implement an embodiment of the present invention (e.g. code detailed above). Disk storage 95 provides non-volatile storage for computer software instructions 92 and data 94 used to implement an embodiment of the present invention. Central processor unit 84 is also attached to system bus 79 and provides for the execution of computer instructions.

In some embodiments, the processor routines 92 and data 94 are a computer program product (generally referenced 92), including a computer readable medium (e.g., a removable storage medium such as one or more DVD-ROM's, CD-ROM's, diskettes, tapes, etc.) that provides at least a portion of the software instructions for the invention system. Computer program product 92 can be installed by any suitable software installation procedure, as is well known in the art. In another embodiment, at least a portion of the software instructions may also be downloaded over a cable, communication and/or wireless connection. In other embodiments, the invention programs are a computer program propagated signal product 107 embodied on a propagated signal on a propagation medium (e.g., a radio wave, an infrared wave, a laser wave, a sound wave, or an electrical wave propagated over a global network such as the Internet, or other network(s)). Such carrier medium or signals may provide at least a portion of the software instructions for the present invention routines/program 92.

In alternate embodiments, the propagated signal is an analog carrier wave or digital signal carried on the propagated medium. For example, the propagated signal may be a digitized signal propagated over a global network (e.g., the Internet), a telecommunications network, or other network. In one embodiment, the propagated signal is a signal that is transmitted over the propagation medium over a period of time, such as the instructions for a software application sent in packets over a network over a period of milliseconds, seconds, minutes, or longer. In another embodiment, the computer readable medium of computer program product 92 is a propagation medium that the computer system 50 may receive and read, such as by receiving the propagation medium and identifying a propagated signal embodied in the propagation medium, as described above for computer program propagated signal product.

Generally speaking, the term “carrier medium” or transient carrier encompasses the foregoing transient signals, propagated signals, propagated medium, storage medium and the like.

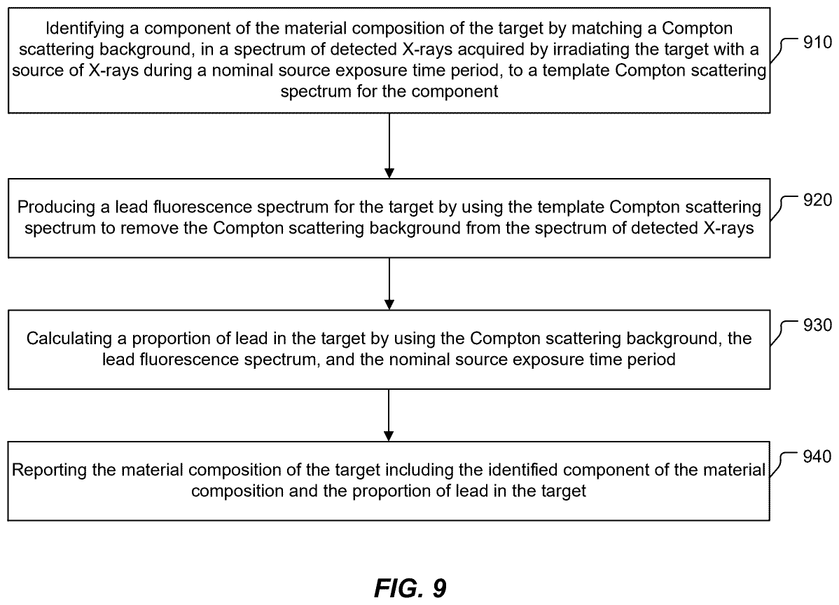

In other embodiments, the program product 92 may be implemented as a so-called Software as a Service (SaaS), or other installation or communication supporting end-users FIG. 9 is a flow diagram illustrating a first generalized method of determining material composition of a target according to an embodiment. The target may be a pipe, as an example. At 910, the method includes identifying a component of the material composition of the target by matching a Compton scattering background, in a spectrum of detected X-rays acquired by irradiating the target with a source of X-rays during a nominal source exposure time period, to a template Compton scattering spectrum for the component. At 920, the method includes producing a lead fluorescence spectrum for the target by using the template Compton scattering spectrum to remove the Compton scattering background from the spectrum of detected X-rays. At 930, the method includes calculating a proportion of lead in the target by using the Compton scattering background, the lead fluorescence spectrum, and the nominal source exposure time period. At 940, the method includes reporting the material composition of the target including the identified component of the material composition and the proportion of lead in the target.

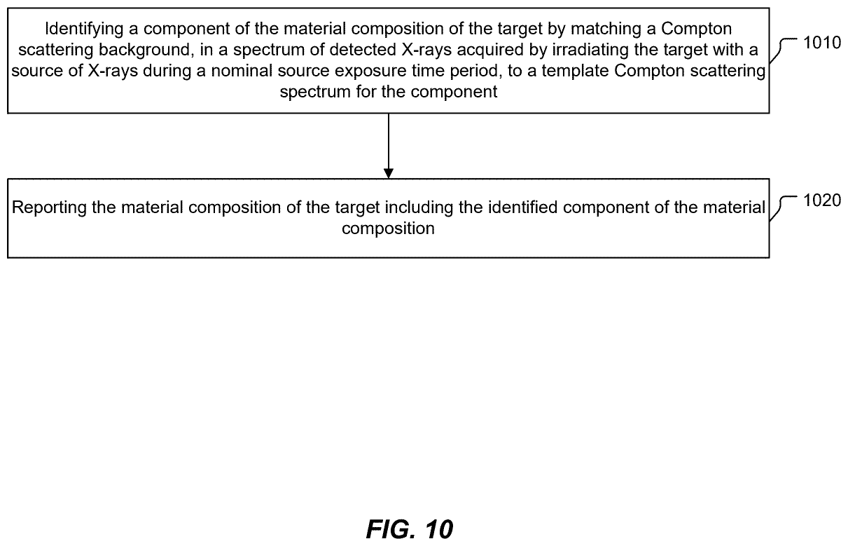

FIG. 10 is a flow diagram illustrating a second generalized method of determining material composition of a target according to an alternative embodiment. The target may be a pipe, as an example. At 1010, the method includes identifying a component of the material composition of the target by matching a Compton scattering background, in a spectrum of detected X-rays acquired by irradiating the target with a source of X-rays during a nominal source exposure time period, to a template Compton scattering spectrum for the component. At 1020, the method includes reporting the material composition of the target including the identified component of the material composition.

In particular implementations, each of the methods illustrated in FIG. 9 and FIG. 10 may further include any of the stages, elements, analyses included within the other, included within the method according to the embodiment of FIG. 3, included in other elements of the description above or the drawings, or described as part of the implementation clauses hereinafter. These implementations will be understood by those of ordinary skill in the art of X-ray scanning and related material analysis in view of the drawings and description herein. Further, each of the methods illustrated in FIG. 9 and FIG. 10 may be implemented in an X-ray scanning device such as that illustrated in FIG. 2. Each of the methods may be implemented in form of, or based on, instructions stored in the non-transitory computer-readable storage media described herein. An example of such storage medium is the disk storage 95 illustrated in FIG. 8. The instructions may cause the CPU 84, for example, to perform steps of the methods in any of their implementations, for example.

### Implementation Clauses

Implementation examples are provided in the following numbered clauses. The numbered clauses represent some embodiments of the present invention and potential claims. (The actual claims are provided at the end of this application.) These clauses form a part of the written description of this application. Accordingly, subject matter of the following clauses may be presented as claims in later proceedings involving this application or any application claiming priority based on this application. Inclusion of such clauses should not be construed to mean that the claims do not cover the subject matter of the clauses. Thus, a decision to not present these clauses as claims in later proceedings should not be construed as a donation of the subject matter to the public.

Without limitation, potential subject matter that may be claimed includes:

Clause 1. A method of determining material composition of a target, the method comprising:
identifying a component of the material composition of the target by matching a Compton scattering background, in a spectrum of detected X-rays acquired by irradiating the target with a source of X-rays during a nominal source exposure time period, to a template Compton scattering spectrum for the component;
producing a lead fluorescence spectrum for the target by using the template Compton scattering spectrum to remove the Compton scattering background from the spectrum of detected X-rays;
calculating a proportion of lead in the target by using the Compton scattering background, the lead fluorescence spectrum, and the nominal source exposure time period; and
reporting the material composition of the target including the identified component of the material composition and the proportion of lead in the target.

Clause 2. The method of clause 1, wherein the target is a pipe.

Clause 3. The method of clause 2, wherein the component is an outer shell of the pipe.

Clause 4. The method of clause 1, wherein the component is selected from the group consisting of: copper, steel, galvanized steel, brass, lead, lead alloy, and combinations thereof.

Clause 5. The method of clause 1, wherein identifying the component of the material composition of the target, producing a lead X-ray fluorescence (XRF) spectrum for the target, calculating the proportion of lead in the target, and reporting the material composition of the target are performed by a handheld device.

Clause 6. The method of clause 1, wherein reporting the material composition of the target includes displaying, on a display screen, information selected from the group consisting of: the material composition, information about the target derived from the material composition, and combinations thereof.

Clause 7. The method of clause 6, wherein the target is a pipe, and wherein reporting the material composition of the target includes displaying the information about the target derived from the material positions, the information selected from the group consisting of: a type of the pipe, a structure of the pipe, and combinations thereof.

Clause 8. The method of clause 1, wherein reporting the material composition of the target includes outputting the material composition to a computer subsystem for derivation of further information about the target based on the material composition.

Clause 9. The method of clause 1, further including acquiring the spectrum of detected X-rays by irradiating the target with the radioactive source during the nominal source exposure time period.

Clause 10. The method of clause 1, wherein matching the Compton scattering background to the template Compton scattering spectrum for the component includes selecting the template Compton scattering spectrum as a best match from a set of template Compton scattering spectra corresponding to a set of respective possible components.

Clause 11. The method of clause 1, wherein using the Compton scattering background includes determining an integrated sum of detected X-rays represented in the Compton scattering background.

Clause 12. The method of clause 1, wherein using the lead fluorescence spectrum includes determining an integrated sum of detected X-rays represented in the lead fluorescence spectrum.

Clause 13. The method of clause 1, wherein calculating a proportion of lead in the target includes calculating a percentage of lead by weight.

Clause 14. A method of determining material composition of a target, the method comprising:
identifying a component of the material composition of the target by matching a Compton scattering background, in a spectrum of detected X-rays acquired by irradiating the target with a source of X-rays during a nominal source exposure time period, to a template Compton scattering spectrum for the component; and
reporting the material composition of the target including the identified component of the material composition.

Clause 15. The method of clause 14, wherein an X-ray fluorescence (XRF) for the target is produced by detecting XRF peaks emitted by an atomic element in the target, that lie on top of the Compton scattering background in the spectrum of detected X-rays.

Clause 16. The method of clause 15, wherein a proportion of an atomic element in the target is calculated by using at least one of the Compton scattering background, the fluorescence spectrum, and the nominal source exposure time period;
reporting the proportion of the atomic element of the target including the identified atomic element.

Clause 17. The method of clause 14, wherein the target is a pipe.

Clause 18. The method of clause 14, wherein the component is selected from the group consisting of: copper, steel, galvanized steel, brass, lead, lead alloy, plastic, and combinations thereof.

Clause 19. The method of clause 15 or clause 16, wherein the atomic element is lead.

Clause 20. The methods of clause 14 or clause 15, wherein identifying the component of the material composition of the target, producing a fluorescence spectrum for the target, calculating the proportion of atomic elements in the target, and reporting the material composition of the target are performed by a handheld device.

Clause 21. The method of clause 14, wherein reporting the material composition of the target includes displaying, on a display screen, information selected from the group consisting of: the material composition, the atomic elemental composition, and combinations thereof.

Clause 22. The method of any of clauses 14-21, wherein the target is a pipe, and wherein reporting the material composition of the target includes displaying the information about the target derived from the material positions, the information selected from the group consisting of: a type of the pipe, a structure of the pipe, and combinations thereof.

Clause 23. The method of clause 14, wherein reporting the material composition of the target includes outputting the material composition to a computer subsystem for derivation of further information about the target based on the material composition.

Clause 24. The method of clause 16, wherein reporting the elemental composition of the target includes outputting the elemental composition to a computer subsystem for derivation of further information about the target based on the elemental composition.

Clause 25. The method of clause 14, further including acquiring the spectrum of detected X-rays by irradiating the target with the radioactive source during the nominal source exposure time period.

Clause 26. The method of clause 14, wherein matching the Compton scattering background includes selecting the template with the best match from a set of pre-stored template Compton scattering spectra corresponding to a set of respective possible components.

Clause 27. The method of clause 16, wherein using the Compton scattering background includes determining an integrated sum of detected X-rays represented in the Compton scattering background.

Clause 28. The method of clause 16, wherein using the fluorescence spectrum includes determining an integrated sum of detected X-rays represented in the fluorescence spectrum.

Clause 29. The method of clause 16, wherein calculating a proportion of an atomic element in the target includes calculating a percentage of the atomic element by weight.

Clause 30. A non-transitory computer-readable storage medium storing instructions that, when executed by a computer, cause the computer to perform a method for determining material composition of a target, the method comprising:
a) identifying a component of the material composition of the target by matching a Compton scattering background, in a spectrum of detected X-rays acquired by irradiating the target with a source of X-rays during a nominal source exposure time period, to a template Compton scattering spectrum for the component;
b) producing a lead fluorescence spectrum for the target by using the template Compton scattering spectrum to remove the Compton scattering background from the spectrum of detected X-rays;
c) calculating a proportion of lead in the target by using the Compton scattering background, the lead fluorescence spectrum, and the nominal source exposure time period; and
d) reporting the material composition of the target including the identified component of the material composition and the proportion of lead in the target.

Clause 31. A non-transitory computer-readable storage medium storing instructions that, when executed by a computer, cause the computer to perform a method for determining material composition of a target, the method comprising:
a) identifying a component of the material composition of the target by matching a Compton scattering background, in a spectrum of detected X-rays acquired by irradiating the target with a source of X-rays during a nominal source exposure time period, to a Compton scattering spectrum for the component; and
b) reporting the material composition of the target including the identified component of the material composition.

### Further Considerations

While example embodiments have been particularly shown and described, it will be understood by those skilled in the art that various changes in form and details may be made therein without departing from the scope of the embodiments encompassed by the appended claims.

What is claimed is:

1. A method of determining material composition of a target, the method comprising:
identifying a component of the material composition of the target by matching a Compton scattering background, in a spectrum of detected X-rays acquired by irradiating the target with a source of X-rays during a nominal source exposure time period, to a template Compton scattering spectrum for the component;
producing a lead fluorescence spectrum for the target by using the template Compton scattering spectrum to remove the Compton scattering background from the spectrum of detected X-rays;
calculating a proportion of lead in the target by using the Compton scattering background, the lead fluorescence spectrum, and the nominal source exposure time period; and
reporting the material composition of the target including the identified component of the material composition and the proportion of lead in the target.

2. The method of claim 1, wherein the target is a pipe.

3. The method of claim 2, wherein the component is an outer shell of the pipe.

4. The method of claim 1, wherein the component is selected from the group consisting of:
copper, steel, galvanized steel, brass, lead, lead alloy, and combinations thereof.

5. The method of claim 1, wherein identifying the component of the material composition of the target, producing a lead X-ray fluorescence (XRF) spectrum for the target, calculating the proportion of lead in the target, and reporting the material composition of the target are performed by a handheld device.

6. The method of claim 1, wherein reporting the material composition of the target includes displaying, on a display screen, information selected from the group consisting of: the material composition, information about the target derived from the material composition, and combinations thereof.

7. The method of claim 6, wherein the target is a pipe, and wherein reporting the material composition of the target includes displaying the information about the target derived from the material positions, the information selected from the group consisting of: a type of the pipe, a structure of the pipe, and combinations thereof.

8. The method of claim 1, wherein reporting the material composition of the target includes outputting the material composition to a computer subsystem for derivation of further information about the target based on the material composition.

9. The method of claim 1, further including acquiring the spectrum of detected X-rays by irradiating the target with the radioactive source during the nominal source exposure time period.

10. The method of claim 1, wherein matching the Compton scattering background to the template Compton scattering spectrum for the component includes selecting the template Compton scattering spectrum as a best match from a set of template Compton scattering spectra corresponding to a set of respective possible components.

11. The method of claim 1, wherein using the Compton scattering background includes determining an integrated sum of detected X-rays represented in the Compton scattering background.

12. The method of claim 1, wherein using the lead fluorescence spectrum includes determining an integrated sum of detected X-rays represented in the lead fluorescence spectrum.

13. The method of claim 1, wherein calculating a proportion of lead in the target includes calculating a percentage of lead by weight.

14. A method of determining material composition of a target, the method comprising:
identifying a component of the material composition of the target by matching a Compton scattering background, in a spectrum of detected X-rays acquired by irradiating the target with a source of X-rays during a nominal source exposure time period, to a template Compton scattering spectrum for the component; and
reporting the material composition of the target including the identified component of the material composition.

15. The method of claim 14, wherein an X-ray fluorescence (XRF) spectrum for the target is produced by detecting XRF peaks, emitted by an atomic element in the target, that lie on top of the Compton scattering background in the spectrum of detected X-rays.

16. The method of claim 15, wherein a proportion of an atomic element in the target is calculated by using at least one of the Compton scattering background, the fluorescence spectrum, and the nominal source exposure time period;
reporting the proportion of the atomic element of the target including the identified atomic element.

17. The method of claim 14, wherein the target is a pipe.

18. The method of claim 14, wherein the component is selected from the group consisting of: copper, steel, galvanized steel, brass, lead, lead alloy, plastic, and combinations thereof.

19. The method of claim 15, wherein the atomic element is lead.

20. The methods of claim 14, wherein identifying the component of the material composition of the target, producing a fluorescence spectrum for the target, calculating the proportion of atomic elements in the target, and reporting the material composition of the target are performed by a handheld device.

21. The method of claim 14, wherein reporting the material composition of the target includes displaying, on a display screen, information selected from the group consisting of: the material composition, the atomic elemental composition, and combinations thereof.

22. The method of claim 14, wherein the target is a pipe, and wherein reporting the material composition of the target includes displaying the information about the target derived from the material positions, the information selected from the group consisting of: a type of the pipe, a structure of the pipe, and combinations thereof.

23. The method of claim 14, wherein reporting the material composition of the target includes outputting the material composition to a computer subsystem for derivation of further information about the target based on the material composition.

24. The method of claim 16, wherein reporting the elemental composition of the target includes outputting the elemental composition to a computer subsystem for derivation of further information about the target based on the elemental composition.

25. The method of claim 14, further including acquiring the spectrum of detected X-rays by irradiating the target with the radioactive source during the nominal source exposure time period.

26. The method of claim 14, wherein matching the Compton scattering background includes selecting the template with the best match from a set of pre-stored template Compton scattering spectra corresponding to a set of respective possible components.

27. The method of claim 16, wherein using the Compton scattering background includes determining an integrated sum of detected X-rays represented in the Compton scattering background.

28. The method of claim 16, wherein using the fluorescence spectrum includes determining an integrated sum of detected X-rays represented in the fluorescence spectrum.

29. The method of claim 16, wherein calculating a proportion of an atomic element in the target includes calculating a percentage of the atomic element by weight.

30. A non-transitory computer-readable storage medium storing instructions that, when executed by a computer, cause the computer to perform a method for determining material composition of a target, the method comprising:
a) identifying a component of the material composition of the target by matching a Compton scattering background, in a spectrum of detected X-rays acquired by irradiating the target with a source of X-rays during a nominal source exposure time period, to a template Compton scattering spectrum for the component;
b) producing a lead fluorescence spectrum for the target by using the template Compton scattering spectrum to remove the Compton scattering background from the spectrum of detected X-rays;
c) calculating a proportion of lead in the target by using the Compton scattering background, the lead fluorescence spectrum, and the nominal source exposure time period; and
d) reporting the material composition of the target including the identified component of the material composition and the proportion of lead in the target.

31. A non-transitory computer-readable storage medium storing instructions that, when executed by a computer, cause the computer to perform a method for determining material composition of a target, the method comprising:
a) identifying a component of the material composition of the target by matching a Compton scattering background, in a spectrum of detected X-rays acquired by irradiating the target with a source of X-rays during a nominal source exposure time period, to a Compton scattering spectrum for the component; and
b) reporting the material composition of the target including the identified component of the material composition.
